(19) United States
(12) Patent Application Publication
(10) Pub. No.: US 2012/0289804 A1
(43) Pub. Date: Nov. 15, 2012

(54) BACK CALIBRATION OF SENSOR DATA

(75) Inventor: Simin Yao, Boonton Township, NJ (US)

(73) Assignee: BAYER HEALTHCARE LLC, Tarrytown, NY (US)

(21) Appl. No.: 13/469,529

(22) Filed: May 11, 2012

Related U.S. Application Data
(60) Provisional application No. 61/484,985, filed on May 11, 2011.

(51) Int. Cl. A61B 5/1455; G06F 19/00

(52) U.S. Cl. 600/365; 702/104; 702/19; 600/309

(57) ABSTRACT

Methods, apparatuses and systems for back calibration of data from a continuous sensor are provided. The continuous sensor may be calibrated periodically by comparing raw sensor values from the sensor to sensor values obtained from a second sensor, such as a blood glucose meter (BGM). Each calibration may produce a calibration factor. In an aspect, the calibration factor may be applied to sensor values acquired prior to the calibration (i.e., back calibration). In a further aspect, a first calibration and a second calibration may be applied to raw sensor values acquired at a time point between the first calibration and the second calibration. The first and second calibrations may be applied to the raw sensor values by weighted averaging according to the proximity of the first and second calibrations to the acquisition time of the raw sensor value.

### CROSS REFERENCE TO RELATED APPLICATIONS

The present application claims priority to U.S. Provisional Patent Application No. 61/484,985, filed May 11, 2011, entitled “BACK CALIBRATION METHOD FOR SENSOR,” the entire disclosure of which is hereby incorporated by reference in its entirety.

### TECHNICAL FIELD

Embodiments herein relate to the field of sensors, and, more specifically, to back calibration of sensor data.

### BACKGROUND

Continuous sensors, such as continuous glucose monitoring (CGM) sensors, are used to measure data continuously, e.g., in a continuous data stream and/or sampled data points over a time interval. The data must be calibrated to ensure proper conversion of raw data from the sensor into a corresponding parameter measurement, such as a glucose level. The calibration is often done by taking one or more parameter readings using a single-use sensor, such as a blood glucose meter (BGM), and correlating those values to the CGM data. However, many sensors drift as time passes, so that a value of the continuous sensor raw data corresponding to a given parameter value may change over time. Accordingly, the continuous sensor must be re-calibrated periodically. However, calibrations are only used for future measurements of the continuous sensor. This may cause some past data to be unusable if the previous calibration is no longer valid, and may cause the calibrated sensor data to be inaccurate.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 illustrates a timeline in accordance with various aspects described herein;

FIG. 2 illustrates a timeline in accordance with various aspects described herein; and

FIG. 3 illustrates an example sensor system in accordance with various aspects described herein.

### DETAILED DESCRIPTION OF THE DISCLOSURE

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope is defined by the appended claims and their equivalents.

Various operations may be described as multiple discrete operations in turn, in a manner that may be helpful in understanding the disclosure; however, the order of description should not be construed to imply that these operations are order dependent.

The description may use perspective-based descriptions such as up/down, back/front, and top/bottom. Such descriptions are merely used to facilitate the discussion and are not intended to restrict the scope of the disclosure.

The terms “coupled” and “connected,” along with their derivatives, may be used. It should be understood that these terms are not intended as synonyms for each other. Rather, in particular aspects, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

In various embodiments, methods, apparatuses, and systems for back calibration of sensor data are provided. A computing device may be endowed with one or more components of the disclosed apparatuses and/or systems and may be employed to perform one or more methods as disclosed herein.

Various aspects of a method of back calibration of data from an analyte sensor are provided. Back calibration may include applying a calibration factor obtained/derived from a calibration to one or more raw sensor values acquired from the sensor prior to the calibration. The back calibration produces a calibrated sensor value based on the raw sensor value and the calibration factor. The back calibration may modify an existing calibrated sensor value, and/or other data that depends on the calibrated sensor values, such as trend data that measures the change in calibrated sensor values over time.

In an aspect, the analyte sensor may be a medical sensor, such as a continuous glucose monitor (CGM), that measures a parameter of a patient, such as glucose level. The sensor may be calibrated periodically by comparing data from the sensor to data obtained from a second sensor, such as a blood glucose meter (BGM). Each calibration may produce a calibration factor. The calibration factor may be a mathematical function, such as a multiplication factor, that converts the raw sensor values to calibrated sensor values that represent the measured parameter of the patient.

In an aspect, the calibration factor may be applied to a raw sensor value acquired at a time point prior to the calibration (i.e., back calibration). In a further aspect, the calibration factor may be applied to raw sensor values acquired at a time point after the calibration (i.e., forward calibration).

In an aspect, a first calibration and a second calibration may be performed, yielding a first calibration factor and a second calibration factor, respectively. The second calibration may be performed after the first calibration. In an aspect, raw sensor values acquired at time points between the first calibration and second calibration may be calibrated by applying the first calibration factor and/or the second calibration factor. In some cases, the first and second calibration factors may be applied by a weighted average according to the temporal proximity of the first and second calibrations to the time point of the raw sensor value.

In an aspect, the calibration factor may be applied only to raw sensor values acquired within a certain time interval before and/or after the calibration. For example, a calibration factor acquired later in time than the raw sensor value may be applied to the raw sensor value only if the raw sensor value was acquired within a back calibration interval prior to the time the calibration was performed. The back calibration interval is a time period within which the calibration factor may be applied to raw sensor values acquired prior to the calibration with a suitable level of accuracy and/or reliability.

Similarly, a calibration factor acquired earlier in time than the raw sensor value may be applied to the raw sensor value only if the raw sensor value was acquired within a forward calibration interval after the time the calibration was performed. The forward calibration interval is a time period within which the calibration factor may be applied to raw sensor values acquired after the calibration with a suitable level of accuracy and/or reliability. The back calibration interval and forward calibration interval may be the same or different lengths of time.

In an aspect, sensor values represent conditions in the body. For example, the raw sensor value from a CGM and/or BGM is indicative of the glucose level in the body of the patient. In some cases, the raw sensor values from the CGM may represent the glucose level that was present in the blood/tissue at a time point prior to the time point the raw sensor value is received/displayed. This difference in time is referred to herein as an offset time, and is reflective of delays that may be introduced because of glucose movement through the sensor membranes, data processing time, etc. In an aspect, the offset time may be a few minutes, such as about 4-5 minutes. In contrast, a BGM may measure the glucose level in the blood directly. In an aspect, the offset time may be taken into account in performing the calibrations and/or calibrating the raw sensor values. For example, in performing a calibration, a BGM value may be compared to a CGM value obtained later in time than the BGM value, such as delayed by the amount of the offset time.

In an aspect, a sensor system is provided including the CGM, the BGM, and a calibration module. The calibration module may be packaged in the same housing with the CGM and/or BGM, or the calibration module may be included in a monitoring unit remotely disposed from the CGM and BGM. The calibration module may be communicatively coupled with the CGM/BGM, such as by wired and/or wireless (e.g., radio frequency) communication.

The calibration module may receive the raw sensor data from the CGM and the BGM data from the BGM. The calibration module uses the CGM/BGM data to produce one or more calibration factors, such as first and second calibration factors, as described above. The calibration module then applies the calibration factors to the raw sensor data, as described above, to produce calibrated sensor data.

In an aspect, the calibration module may also transmit commands and/or other information to the CGM and/or BGM. For example, the calibration module may transmit a message when a new calibration is needed.

In an aspect, the CGM, BGM, calibration module, and/or other monitoring unit may include a display to present data, alerts, and/or other information to the user (e.g., the patient and/or a caregiver). The display may show any suitable data, such as the raw sensor data, calibrated sensor data, and/or trend data. Additionally, the display may show information related to the calibrations, such as the time of the most recent calibration and/or the time remaining until the next calibration is required. In some cases, the system may activate an alert to tell the user that a calibration is required or is about to be required. For example, the system may require a calibration at or near the time the forward calibration interval from the most recent calibration expires. Alternatively, the system may take into account the backward calibration interval of the next calibration when setting a time for the next calibration. In this case, the time between calibrations may be longer. The display may include any suitable means for presenting information to the user, such as a screen (e.g., a liquid crystal diode (LCD) screen), a touchscreen, a clock, and/or one or more lights (e.g., light emitting diodes (LEDs)).

In various aspects, a monitoring unit may be any suitable device, such as a personal data assistant, mobile phone, personal computer, laptop computer, tablet computer, a watch, and/or a dedicated computing device for the sensor system.

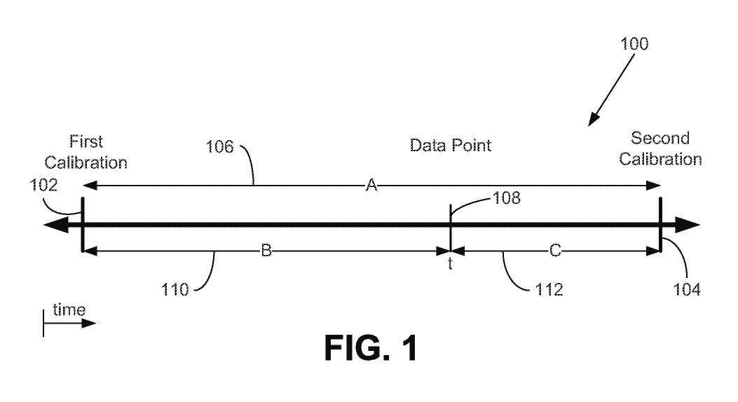

FIG. 1 shows a timeline 100 representing aspects of a back calibration method. As shown, a first calibration is performed at a first calibration time 102 and a second calibration is performed at a second calibration time 104. The first calibration produces a first calibration factor, Y, and the second calibration produces a second calibration factor, Z. The second calibration time 104 is later in time than the first calibration time 102 by a first time interval 106 (A). A raw sensor value of a continuous sensor is acquired at a time point 108 (t). The time point 108 is later in time than the first calibration time by a second time interval 110 (B), and earlier in time than the second calibration time by a third time interval 112 (C), where the second time interval 110 and third time interval 112 combine to equal the first time interval 106.

The first calibration factor and second calibration factor are both applied to the raw sensor value to produce a calibrated sensor value for the time point 108. In an aspect, the first calibration factor and second calibration factor may be applied to the data point according to a weighted average based on a ratio of the second time interval 110 relative to the third time interval 112.

For example, assume that a calibrated sensor value for a given time t and calibration factor f is represented by the function Cal(t, f). In an aspect, the final calibrated sensor value is a weighted average of a first calibrated value (Cal(t,Y)) using only the first calibration factor and a second calibrated value (Cal(t, Z)) using only the second calibration factor. In an aspect, the first calibrated value and second calibrated value are averaged according to a ratio of the second time interval relative to the third time interval, so that the calibration that occurred in closer temporal proximity to the time point at which the data was acquired makes up a greater component of the final reading. For a linear average, the final calibrated sensor value at the time point (Cal(t)) is calculated according to the function Cal(data time, calibration factor) (Eq. 1):

Cal(t)=Cal(t,Y)*C/A+Cal(t,Z)*B/A

As an example, assume the first time interval, A, between the first calibration time and the second calibration time is 24 hours, and the raw sensor value was acquired 16 hours after the first calibration time and 8 hours prior to the second calibration time. In this scenario, the final calibrated sensor value is the sum of the calibrated sensor value calculated using the first calibration factor multiplied by 8/24 (⅓) and the calibrated sensor value calculated using the second calibration factor multiplied by 16/24 (⅔).

In an aspect, the weighted average accounts for a drift characteristic of the continuous sensor that represents how a value of the continuous sensor raw data that corresponds to a given value of the measured parameter changes over time. A linear average, as shown in Eq. 1, may be appropriate for continuous sensors that drift linearly.

It will be apparent that other mathematical methods and/or other orders of operations may be used to arrive at the same or similar results. For example, for some types of calibration factors and/or sensor drift characteristics, the first and second calibration factors may be combined into a final calibration factor prior to being applied to the data point. Following the example above, for a raw sensor value acquired 16 hours after the first calibration factor, Y, and 8 hours prior to the second calibration factor, Z, the final calibration factor, X, may be X=⅓Y+⅔Z.

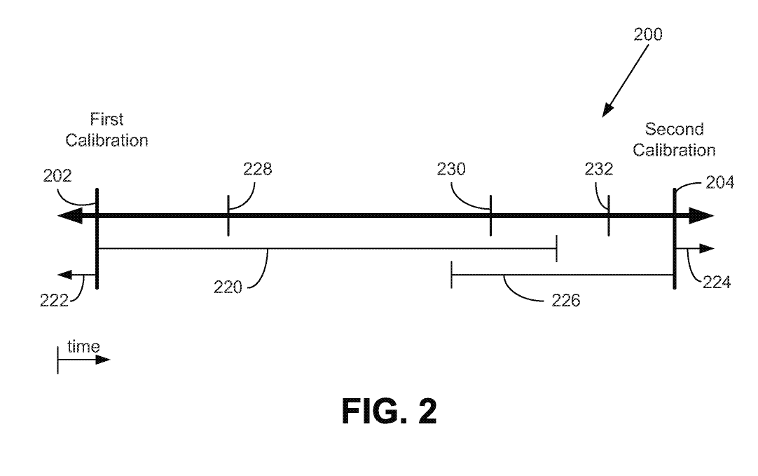

In an aspect, each calibration factor is applied only to raw sensor values acquired within a proximity interval before and/or after the time of the calibration. FIG. 2 illustrates a timeline 200 showing when a first calibration 202 and a second calibration 204 were performed. The first calibration produces a first calibration factor and the second calibration produces a second calibration factor. The first calibration 202 is only applied to raw values acquired within a forward calibration interval 220 after first calibration 202 and/or within a backward calibration interval 222 prior to first calibration 202. Similarly, the second calibration 204 is only applied to raw values acquired within a forward calibration interval 224 after second calibration 204 and/or within a backward calibration interval 226 prior to second calibration 204.

FIG. 2 illustrates the time points of acquisition for three sensor values 228, 230, and 232 acquired between first calibration 202 and second calibration 204. Sensor value 228 was acquired within the forward calibration interval 220 of first calibration 202, but not within the backward calibration interval 226 of second calibration 204. Accordingly, sensor value 228 will be calibrated by applying the first calibration factor, and not the second calibration factor.

Sensor value 232 was acquired after expiration of the forward calibration interval 220 from first calibration 202, but within the backward calibration interval 226 of second calibration 204. Accordingly, sensor value 232 will be calibrated by applying the second calibration factor, and not the first calibration factor.

Sensor value 230 was acquired within the forward calibration interval 220 of first calibration 202 and within the backward calibration interval 226 of second calibration 204. Accordingly, sensor value 230 will be calibrated by applying both the first calibration factor and second calibration factor by weighted averaging, as explained above.

In an aspect, a calibration factor is removed as a component of the final calibrated sensor value before expiration of the forward calibration interval and/or backward calibration interval if an intervening calibration is performed. For example, if the forward calibration interval is twenty-four hours, a calibration factor obtained twenty hours prior to the raw sensor value may no longer be used if another calibration factor was obtained two hours prior to the raw sensor value. Alternatively, both calibration factors may be used to calculate the calibrated sensor value (e.g., by a weighted average).

In an aspect, the back calibration method may use only one calibration factor to calculate the calibrated sensor value. For example, the calibration that is closest in time to the time point of the raw sensor value may be used to calculate the final calibrated sensor value (e.g., either back calibration or forward calibration). Accordingly, the calibration that is further in time from the time point of the raw sensor value may not be used.

In an aspect, calibrated sensor values may be updated if further calibrations are performed. For example, raw sensor data may be initially converted (e.g., in real-time or close to real-time) to calibrated sensor data based on then-available calibrations. If a later calibration is performed, the calibrated sensor data may be updated based on the later calibration.

The use of back calibration, as provided herein, may allow continuous sensor data to be used when a calibration performed prior to acquiring the raw sensor value has expired (i.e., the forward calibration interval has passed). The sensor value may be calibrated using a calibration performed later in time than the acquisition time point of the raw sensor value, and within the backward calibration interval from the acquisition time point. This may allow more sensor data to be used and/or allow for a longer time between calibrations.

Furthermore, back calibration may improve the accuracy of the final sensor reading by using multiple calibrations. The multiple calibrations may account for the drift characteristic of the continuous sensor and/or provide additional calibration data to improve the accuracy of the final sensor readings.

In an aspect, two or more calibration factors may be used as a component of the final calibrated sensor value. For example, a plurality of calibrations performed prior to acquiring the raw sensor value and/or a plurality of calibrations performed after acquiring the raw sensor value may be used to calculate the calibrated sensor value from the raw sensor value. The multiple calibrations may be jointly applied by weighted averaging and/or another suitable method.

Further aspects of the back calibration method may include storing the raw sensor values, storing the calibrated sensor values, and/or monitoring for a calibration performed within the backward calibration interval from the acquisition time of the sensor values. If a calibration occurs within the backward calibration interval from the acquisition time of one or more of the stored sensor values, the calibration may be applied to the stored sensor values as described above.

Aspects may also include apparatuses and/or systems for carrying out the back calibration method. The apparatuses and/or systems may include a continuous sensor and/or a computing device, such as a microcontroller, that receives raw sensor values from the continuous sensor. The microcontroller may also receive data from a second sensor and calculate one or more calibration factors based on the data from the second sensor and the raw sensor values from the continuous sensor. The microcontroller may use the one or more calibration factors to convert the raw sensor values from the continuous sensor to calibrated sensor values, as described above.

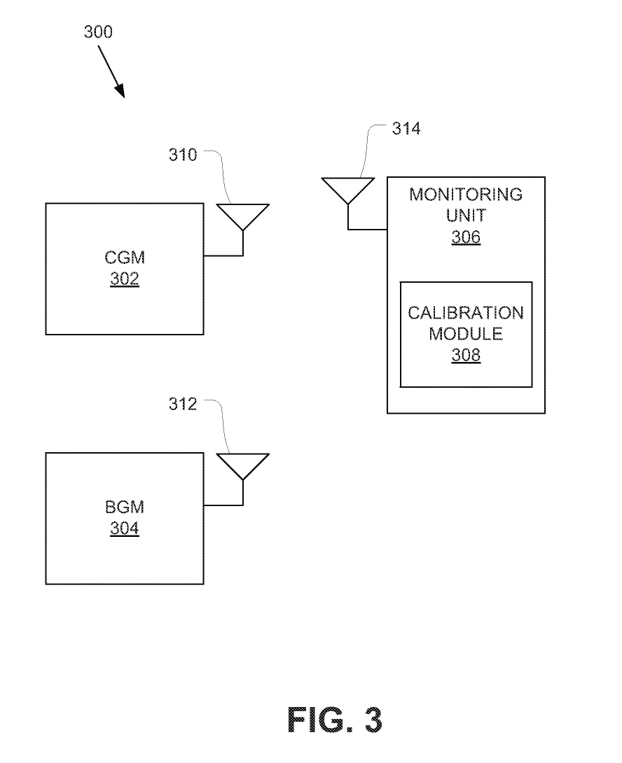

FIG. 3 shows a sensor system 300 including a CGM 302, a BGM 304, and a monitoring unit 306. The monitoring unit 306 includes a calibration module 308. CGM 302 is coupled to a body of a patient to measure glucose levels in a bloodstream of the patient. The CGM 302 produces raw sensor data based on the glucose level in the patient's body. The CGM 302 transmits the raw sensor data to the monitoring unit 306. The CGM 302 transmits the raw sensor data to the monitoring unit 306 wirelessly using an antenna 310 (e.g., over radio frequency (RF)). Alternatively, the CGM 302 may transmit the raw sensor data to the monitoring unit 306 by other means, such as a wired connection. In some cases, the CGM 302 and monitoring unit 306 may be included in the same package.

BGM 304 periodically takes measurements of blood glucose level and transmits the data to monitoring unit 306 using an antenna 312. Alternatively, or additionally, the BGM 304 may transmit the BGM data to the monitoring unit 306 by other means, such as a wired connection and/or user input. In some situations, the CGM 302 and/or the BGM 304 may be included in the same housing/device as monitoring unit 306.

The monitoring unit 306 receives the raw sensor values from the CGM 302 and the BGM data from the BGM 304 via an antenna 314. The calibration module 308 uses the BGM data to produce one or more calibration factors, such as first and second calibration factors, as described above. The calibration module 308 then applies the calibration factors to the raw sensor data, as described above, to produce calibrated sensor data.

In an aspect, the monitoring unit 306 may also transmit commands and/or other information to the CGM 302 and/or BGM 304. For example, the monitoring unit 306 may transmit a message when a new calibration is needed.

In an aspect, the CGM 302 is designed to be coupled to the body of the patient for continuous monitoring of glucose level. The CGM 302 may take continuous measurements and/or periodic measurements (e.g., every few minutes).

In an aspect, the CGM 302 may include a sensor assembly that includes a glucose sensor, and an electronics assembly that includes electronics to process the signal from the sensor and/or transmit the sensor data to the monitoring unit 306. In an aspect, the sensor assembly is designed to be used for a relatively short period of time, such as about 1-2 weeks, and then replaced. In contrast, the electronics assembly is designed to be used for a relatively longer period of time. Thus, when it is time to replace the sensor assembly, the sensor assembly is removed from the CGM 302 and a new sensor assembly is coupled to the electronics assembly. The sensor assembly may also be referred to as the disposable sensor assembly, and the electronics assembly may also be referred to as the reusable sensor assembly. In an alternative aspect, the electronics assembly may include the calibration module 308.

The monitoring unit 306 may be any suitable device, such as a computing device, such as a personal data assistant, mobile phone, personal computer, laptop computer, tablet computer, a watch, and/or a dedicated computing device for the sensor system. The monitoring unit 306 may include a display for displaying data and/or messages to the patient and/or a caregiver. For example, the display may show when a BGM measurement is required for calibration and/or the time remaining until the next BGM measurement is required. The display may further display the data from the CGM 302 and/or BGM 304, before and/or after calibration.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

1. A method comprising:
performing, by a computing device, a calibration of an analyte sensor at a calibration time to produce a calibration factor; and
applying, by the computing device, the calibration factor to raw sensor values measured by the analyte sensor within a back calibration interval prior to the calibration time to produce and/or modify calibrated sensor values from the raw sensor values.

2. The method of claim 1, further comprising applying, by the computing device, the calibration factor to raw sensor values measured by the analyte sensor within a forward calibration interval after the calibration time.

3. The method of claim 2, further comprising activating an alert based on an expiration time of the forward calibration interval.

4. The method of claim 1, wherein the calibration factor is a first calibration factor and the calibration time is a first calibration time, and wherein the first calibration factor is applied to the raw sensor values according to a weighted average with a second calibration factor obtained at a second calibration time before the raw sensor values were measured, wherein the first calibration factor and second calibration factor are weighted based on a proximity of the first calibration time and second calibration time, respectively, to a time point of the raw sensor value.

5. The method of claim 1, wherein the computing device is a monitoring unit, and the method further comprising transmitting the raw sensor values from the analyte sensor to the monitoring unit for calibration.

6. The method of claim 1, wherein the analyte sensor is a first analyte sensor, and wherein the performing the calibration includes comparing a first raw sensor value from the first analyte sensor to a second raw sensor value from a second analyte sensor, wherein the second analyte sensor is of a different type from the first analyte sensor.

7. The method of claim 6, wherein the first analyte sensor is a continuous glucose monitor (CGM), and the second analyte sensor is a blood glucose meter (BGM).

8. The method of claim 1, further comprising updating trend data for a time period prior to the calibration time based on the calibration factor.

9. A method comprising:
performing, by a computing device, a first calibration of an analyte sensor to produce a first calibration factor;
performing, by the computing device, a second calibration of the analyte sensor to produce a second calibration factor, the second calibration performed after the first calibration; and
applying, by the computing device, the first calibration factor and the second calibration factor to a raw sensor value measured by the analyte sensor at a time point between the first and second calibration to produce and/or modify a calibrated sensor value for the time point.

10. The method of claim 9, wherein the first calibration factor and second calibration factor are applied to the raw sensor value by weighted averaging.

11. The method of claim 10, wherein the first calibration factor and second calibration factor are weighted according to a proximity of the time point with the first calibration and second calibration, respectively.

12. The method of claim 9, wherein the time point of the raw sensor value is within a forward calibration interval from a first calibration time of the first calibration and within a backward calibration interval from a second calibration time of the second calibration.

13. The method of claim 9, wherein the computing device is a monitoring unit, and the method further comprising transmitting the raw sensor values from the analyte sensor to the monitoring unit for calibration.

14. The method of claim 9, wherein the analyte sensor is a first analyte sensor, and wherein the performing the calibration includes comparing a first raw sensor value from the first analyte sensor to a second raw sensor value from a second analyte sensor, wherein the second analyte sensor is of a different type from the first analyte sensor.

15. The method of claim 9, wherein the analyte sensor is a continuous glucose monitor (CGM).

16. A sensor system comprising:
an analyte sensor configured to produce raw sensor values that depend on a concentration of an analyte in a body;
a calibration module communicatively coupled to the analyte sensor and configured to receive the raw sensor values, the calibration module configured to:
perform a calibration of the analyte sensor at a calibration time to produce a calibration factor; and
apply the calibration factor to the raw sensor values produced by the analyte sensor within a back calibration interval prior to the calibration time to produce and/or modify calibrated sensor values from the raw sensor values.

17. The system of claim 16, wherein the calibration module is further configured to apply the calibration factor to the raw sensor values produced by the analyte sensor within a forward calibration interval after the calibration time.

18. The system of claim 17, wherein the calibration module is further configured to activate an alert based on an expiration time of the forward calibration interval.

19. The system of claim 16, wherein the calibration factor is a first calibration factor and the calibration time is a first calibration time, and wherein calibration module is configured to apply the first calibration factor to the raw sensor values according to a weighted average with a second calibration factor obtained at a second calibration time before the raw sensor values were produced, wherein the first calibration factor and second calibration factor are weighted based on a proximity of the first calibration time and second calibration time, respectively, to a time point of the raw sensor value.

20. The system of claim 16, wherein the analyte sensor is a first analyte sensor, and wherein the performing the calibration includes comparing a first raw sensor value from the first analyte sensor to a second raw sensor value from a second analyte sensor, wherein the second analyte sensor is of a different type from the first analyte sensor.

21. The system of claim 20, wherein the first analyte sensor is a continuous glucose monitor (CGM), and the second analyte sensor is a blood glucose meter (BGM).

22. The system of claim 16, wherein the calibration module is included in a monitoring unit remotely disposed from the analyte sensor.

23. The system of claim 16, wherein the analyte sensor and the calibration module are included in a same housing.
